(19) United States
(12) Patent Application Publication
(10) Pub. No.: US 2017/0169164 A1
(43) Pub. Date: Jun. 15, 2017

(54) PHENOTYPE-BASED CHEMICAL SCREENS

(71) Applicants: The General Hospital Corporation, d/b/a Massachusetts General Hospital, Boston, MA (US); Teleos Therapeutics, LLC, Concord, MA (US)

(72) Inventors: David Kokel, Oakland, CA (US); Randall T. Peterson, Belmont, MA (US); Giancarlo Bruni, Boulder, CO (US); Andrea Velenich, Somerville, MA (US)

(21) Appl. No.: 15/336,152

(22) Filed: Oct. 27, 2016

Related U.S. Application Data
(60) Provisional application No. 62/267,122, filed on Dec. 14, 2015.

(51) Int. Cl. G06F 19/28; A61K 49/00

(52) U.S. Cl. CPC G06F 19/28; A61K 49/0008

(57) ABSTRACT

Embodiments of the present invention provide materials and methods for performing phenotype-based chemical screens. Embodiments disclosed herein pertain to the use of a battery of high-throughput zebrafish behavioral assays to generate behavioral profiles. In accordance with these embodiments, responses to various chemical compounds with known and unknown biological functions can be used to generate behavioral profiles. Embodiments also involve generating behavioral profiles based on genetic mutations or environmental perturbations. Establishing databases of behavioral profiles facilitates identification of novel chemicals that phenocopy effects of therapeutic agents and/or that modulate genetic or environmental behavioral profiles, thereby providing a basis for not only assessing the properties of known chemical compounds but also for developing novel treatments for human diseases.

### RELATED APPLICATIONS

This non-provisional patent application claims priority to and the benefit of U.S. Provisional Patent Application Ser. No. 62/267,122, filed Dec. 14, 2015. This application is incorporated herein by reference in its entirety for all purposes.

### STATEMENT REGARDING FEDERALLY FUNDED RESEARCH

Embodiments disclosed herein have been supported in part by National Institutes of Health (NIH) grants K01MH091449, R01AA022583, MH086867, and MH085205. The government has certain rights to embodiments of this invention.

### FIELD

Embodiments of the present invention provide materials and methods for performing phenotype-based chemical screens. Embodiments disclosed herein pertain to the use of a battery of high-throughput zebrafish behavioral assays to generate behavioral profiles. In accordance with these embodiments, responses to various chemical compounds with known and unknown biological functions can be used to generate behavioral profiles. Embodiments also involve generating behavioral profiles based on genetic mutations or environmental perturbations. Embodiments also concern establishing a database of behavioral profiles to facilitate identification of novel chemicals that phenocopy effects of therapeutic agents and/or that modulate genetic or environmental behavioral profiles, thereby providing a basis for developing novel treatments for human diseases.

### BACKGROUND

Mental health disorders, including schizophrenia, depression, bipolar disorder and other psychiatric disorders, affect large numbers of the population with approximately 30% of adults in the United States suffering from some form of mental illness. The economic burden is estimated to be hundreds of billions of dollars each year. Non-psychiatric diseases of the central nervous system (CNS) further increase this economic and health burden. Despite decades of intense CNS drug discovery efforts, many major pharmaceutical companies have shut down or deemphasized their CNS research and development programs. Reasons for this include disappointing late-stage clinical trial results and translational challenges from preclinical to clinical development. For example, success rates for CNS drug candidates in clinical trials are estimated to be less than half that of other therapeutic fields. Today, drug discovery efforts are largely focused on target-based in vitro assays however; phenotype-based approaches have been more likely to identify first-in-class compounds.

Polygenic psychiatric diseases such as schizophrenia have been particularly difficult to treat. Antipsychotic drugs used to treat schizophrenia bind to many receptors in the nervous system, and unlike ‘magic bullet’ drugs (including many antibiotics and some chemotherapeutics that act on single molecular targets) most antipsychotics are thought to act via polypharmacological mechanisms (i.e., on many targets simultaneously). Novel antipsychotics and other polypharmacologic drugs have been difficult to identify using traditional target based assays that focus on isolated receptors in vitro. Polygenic psychiatric disorders, such as schizophrenia, likely require systems-modulating therapeutics, which are difficult to identify without complex in vivo readouts. Given that there are no known biomarkers for most psychiatric disorders, behavior modification is an attractive endpoint for CNS drug screens. However the time, space, and financial resources required for high-throughput (HT) behavioral screening for schizophrenia drug discovery have been prohibitive using traditional animal models.

### SUMMARY

Embodiments of the present invention provide materials and methods for identifying novel therapeutic chemical compounds. Embodiments of the method comprise comparing a quantitative summary of a behavioral profile of a known bioactive chemical compound to a database of quantitative summaries of behavioral profiles, and identifying a chemical compound or chemical compounds from the database of quantitative summaries of behavioral profiles based on phenotypic similarities to the behavior profile of the known bioactive chemical compound. According to embodiments of the present invention, quantitative summaries of behavioral profiles are generated using one or more organisms, wherein the one or more organisms is one or more larval zebrafish, and wherein exposing the at least one larval zebrafish to a chemical compound comprises adding the chemical compound to a well containing liquid medium.

In some embodiments, the method of the present invention includes behavioral profiles comprised of one or more behavioral assays, wherein the behavioral assays comprise the presentation of one or more stimuli. The one or more stimuli comprise one or more of light, sound, vibration, visual patterns, and electrical shocks. Embodiments also include the use of known bioactive chemical compounds having psychoactive properties, and performing the methods as part of an automated high-throughput screening system or platform.

Embodiments of the present invention provide materials and methods for performing high-throughput screens for bioactive chemical compounds. In accordance with these embodiments, methods can include exposing at least one organism to a chemical compound, subjecting at least one organism to one or more behavioral assays and generating a behavioral profile, obtaining a quantitative summary of the behavioral profile and storing the quantitative summary in a database. Further, other embodiments of the methods can include repeating exposing, subjecting, obtaining, and storing steps using a plurality of chemical compounds to generate a database of quantitative summaries of behavioral profiles. Embodiments of the method can further include the use of an automated high-throughput screening platform.

In some embodiments, methods of the present invention can be performed using various organisms, including but not limited to, zebrafish (e.g., Danio rerio), fruit flies (e.g. Drosophila melanogaster), and nematodes (e.g., C. elegans). In certain methods, the organism can be a larval zebrafish, and the method can include exposing the zebrafish to a chemical compound in the well of a 96-well plate containing liquid medium.

In some embodiments, behavioral assays can include presentation of one or more stimuli to the organism. One or more stimuli can include, but are not limited to, one or more of light, sound, vibration, visual patterns, and electrical shocks. In some aspects, the one or more behavioral assays can include presentation of stimuli such as an acoustic stimuli or a pulse of light for certain durations of time and at a certain frequencies, wavelengths or intensities.

In some embodiments, methods of the present invention can include exposing organisms to a plurality of chemical compounds with known and unknown biological functions. In some aspects, the plurality of chemical compounds are contained within a chemical library, and in some cases, the plurality of compounds include compounds with psychoactive properties.

In other embodiments, automated high-throughput screening platforms with which methods can be performed can include software that synchronizes image acquisition when subjecting the at least one organism to the one or more behavioral assay, and the software extracts behavioral data for storage when obtaining the quantitative summary of the behavioral profile. In other aspects, methods can further include analyzing a database of quantitative summaries of behavioral profiles for common phenotypic characteristics. In other cases, methods further include comparing a quantitative summary of a behavioral profile of a known psychoactive chemical compound to a database of quantitative summaries of behavioral profiles to identify chemical compounds with common phenotypic characteristics.

Embodiments of the present invention also provide a system for performing a high-throughput screen for bioactive chemical compounds. The system includes a digital video camera capable of capturing and storing images, a mechanism for presenting one or more stimuli to at least one organism when subjecting the at least one organism to one or more behavioral assays, software that synchronizes the capturing and storing of images when subjecting the at least one organism to one or more behavioral assays to generate one or more behavioral profiles and extracts behavioral data from the one or more behavioral profiles to generate a one or more quantitative summaries.

Unless otherwise defined, all technical and scientific terms used herein have the same meaning as commonly understood by one of ordinary skill in the art to which this disclosure belongs. Although methods and materials similar or equivalent to those described herein can be used to practice the subject matter of the disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the disclosure will be apparent from the following detailed description, and from the claims.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Other features and advantages of the disclosure will be apparent from the following detailed description, and from the claims.

### BRIEF DESCRIPTION OF THE FIGURES

The following drawings form part of the instant specification and are included to further demonstrate certain aspects of particular embodiments herein. The embodiments may be better understood by reference to one or more of these drawings in combination with the detailed description presented herein.

FIG. 1A is a representative image of a behavioral profile comprised of a battery of 10 individual behavioral assays using the zebrafish, according to an embodiment of the present disclosure.

FIG. 1B is a representative image of a heat map depicting the effects of various structurally unrelated antipsychotic chemicals on zebrafish motor activity as compared to controls, according to one embodiment of the present disclosure.

FIG. 2A is a representative image of a histogram depicting the relative similarity of behavioral profiles of zebrafish exposed to various chemicals compared to the behavioral profile of zebrafish exposed to haloperidol, according to one embodiment of the present disclosure.

FIG. 2B is a representative image of a histogram depicting the relative similarity of behavioral profiles of zebrafish exposed to various chemicals compared to the behavioral profile of zebrafish exposed to haloperidol, with the 47 most haloperidol-like chemicals highlighted, according to one embodiment of the present disclosure.

FIG. 2C lists the results of using phenotype-based screening methods (e.g., phenoBlast) to identify chemicals that produce the most haloperidol-like responses in behavioral assays using zebrafish, according to one embodiment of the present disclosure.

FIG. 2D is a representative cladogram depicting the structural relationships (e.g., Tanimoto distance) among novel chemicals that produce haloperidol-like responses in behavioral assays using zebrafish, according to one embodiment of the present disclosure.

FIG. 2E lists the chemical structures of 5 closely related (61%-89% Tanimoto similarity) novel chemicals that produce haloperidol-like responses in behavioral assays using zebrafish, according to one embodiment of the present disclosure.

FIG. 3A is a representative image of a heat map depicting the effects of various novel chemicals that produce haloperidol-like responses in behavioral assays using the zebrafish, according to one embodiment of the present disclosure.

FIG. 3B is a representative image of a heat map depicting the in vitro binding affinities of novel chemicals that produce haloperidol-like responses, as well as unrelated control chemicals, for various human and rodent neurotransmitter receptors, according to one embodiment of the present disclosure.

FIG. 3C is a representative image of a heat map and cladogram (haloperidol-like clustering) depicting the in vitro binding affinity of a novel haloperidol-like chemical (6557321) for various human and rodent neurotransmitter receptors as compared to other antipsychotic chemicals, according to one embodiment of the present disclosure.

FIG. 3D is a representative graph depicting the effects of a novel haloperidol-like chemical (6557321) on the suppression of the psychostimulant effect in a mouse schizophrenia model, according to one embodiment of the present disclosure.

Unless defined otherwise, all technical and scientific terms used herein have the meaning commonly understood by a person skilled in the art to which this disclosure belongs.

### DETAILED DESCRIPTION

Embodiments of the present invention provide materials and methods for performing phenotype-based chemical screens. Embodiments herein pertain to the use of a battery of high-throughput zebrafish behavioral assays to generate behavioral profiles based on responses to various chemical compounds with known and unknown biological functions. Embodiments of the present disclosure also involve establishing a database of behavioral profiles to facilitate the identification of novel chemicals that phenocopy the effects of therapeutic agents.

In some embodiments, methods of the present invention include subjecting organisms to a battery of behavioral assays to generate a behavioral profile. The behavioral assay can be carried out using various experimental parameters, such as exposing the organisms to different stimuli and quantifying the corresponding behavioral response. Any observable and quantifiable behavior can be used as the basis for a behavioral assay. For example, behavioral assays can be based on a photomotor response in which the applied stimulus is a pulse of bright light. Behavioral assays can be based on sleep patterns in which the applied stimulus involves changing the photoperiod. Behavioral assays can be based on habituation in which the applied stimulus involves modulation of high-frequency stimulus trains. Behavioral assays can be based on associative learning and extinction in which the applied stimulus involves the pairing of conditioned and unconditioned stimuli. Behavioral assays can be based on application of a painful stimulus. Behavioral assays can be based on the acoustic startle response in which the applied stimulus is an acoustic stimulus. Behavioral assays can be based on the visual startle response in which the applied stimulus is a visual stimulus.

In some cases, behavioral assays can be based on various combinations of stimuli applied in succession for specific durations and intensities. For example, a behavioral assay can include the application of acoustic stimuli, electric stimuli, visual stimuli, and the like for specific amounts of time and at specific intensities. Stimuli can be applied sequentially in any order or combination, with or without the addition of refractory periods, as one of skill in the art would readily appreciate based on the present disclosure. Various other stimuli can also be applied, and are not limited to those mentioned above, depending on the organism used and the experimental conditions, as one of ordinary skill would readily appreciate based on the present disclosure. In some cases, a stimulus can be applied for 0.1 second, 0.2 seconds, 0.3 seconds, 0.4 seconds, 0.5 seconds, 0.6 seconds, 0.7 seconds, 0.8 seconds, 0.9 seconds, 1 second, 2 seconds, 3 seconds, 4 seconds, 5 seconds, 6 seconds, 7 seconds, 8 seconds, 9 seconds, 10 seconds, 11, seconds, 12 seconds, 13 seconds, 14 seconds, 15 seconds, 16 seconds, 17 seconds, 18 seconds 19 seconds, 20 seconds, 21 seconds, 22 seconds, 23 seconds, 24 seconds, 25 seconds, 26 seconds, 27 seconds, 28 seconds, 29 seconds, 30 seconds, and/or any number of seconds up to 5 minutes. Stimuli can also be applied multiple times in succession for seconds or sub-second intervals.

Light-based stimuli can be applied by any means and can include light delivered at any wavelength that is capable of causing a response in an organism, including but not limited to red light (660 nanometers), violet light (405 nanometers), and/or blue light (450 nanometers). Light-based stimuli can also be applied multiple times in succession for seconds or sub-second intervals, with or without refractory periods. Electrical stimuli can be applied by any means and can include electricity delivered at 1 volt (V), 2V, 3V, 4V, 5V, 6V, 7V, 8V, 9V, 10V, 11V, 12V, 13V, 14V, 15V, 16V, 17V, 18V, 19V, 20V, 21V, 22V, 23V, 24V, 25V, and/or any amount of volts up to 50V. Electrical stimuli can also be applied multiple times in succession for seconds or sub-second intervals, with or without refractory periods. Acoustic stimuli can be applied by any means and can include electricity delivered at 1 volt (V), 2V, 3V, 4V, 5V, 6V, 7V, 8V, 9V, 10V, 11V, 12V, 13V, 14V, 15V, 16V, 17V, 18V, 19V, 20V, 21V, 22V, 23V, 24V, 25V, and/or any amount of volts up to 50V. Electrical stimuli can also be applied multiple times in succession for seconds or sub-second intervals, with or without refractory periods.

Any number of behavioral assays can be used to generate a behavioral profile. A behavioral profile can include a single behavioral assay, 2 behavioral assays, 3 behavioral assays, 4 behavioral assays, 5 behavioral assays, 6 behavioral assays, 7 behavioral assays, 8 behavioral assays, 9 behavioral assays, 10 behavioral assays, and as many as 20 behavioral assays. The number and type of behavioral assays that make up a behavioral profile can readily be determined by one of skill in the art based on the present disclosure, and can be depend on such factors as, for example, the organism being used and the experimental parameters being investigated.

It is often difficult to predict what types of a chemical compounds a particular behavioral assay will identify. A behavioral assay using a particular stimulus may identify compounds with biological functions that do not appear to correlate with the compounds' identified biological activity in a vertebrate animal (e.g., a human). With sufficiently rich behavioral phenotyping, however, the present invention provides ways to identify mechanistic relationships between molecules within a large collection simply on the basis of their phenotypic similarity. This approach, based solely on the behavioral effects produced by various chemicals, can provide an unbiased, structure- and target-independent platform for identifying novel therapeutic compounds.

Embodiments of the present disclosure are applicable to the discovery of therapeutic agents for the treatment of any disease or disorder. Embodiments of the present disclosure are particularly well suited to the discovery of novel therapeutic agents for the treatment of CNS disorders and diseases, due in part to the polygenic nature of these disorders as well as the dearth of known targets underlying most CNS disorders and diseases. CNS disorders can be drug induced, can be attributed to genetic predisposition, infection or trauma, or can be of unknown etiology. Materials and methods of the present disclosure can be used to treat CNS disorders, including but not limited to neuropsychiatric disorders, neurological diseases and mental illnesses, neurodegenerative diseases, behavioral disorders, cognitive disorders, and cognitive affective disorders. There are several CNS disorders whose clinical manifestations have been attributed to CNS dysfunction (e.g., disorders resulting from inappropriate levels of neurotransmitter release, inappropriate properties of neurotransmitter receptors, and or inappropriate interaction between neurotransmitters and neurotransmitter receptors). Several CNS disorders can be attributed to a cholinergic deficiency, a dopaminergic deficiency, an adrenergic deficiency and/or a serotonergic deficiency. CNS disorders of relatively common occurrence include presenile dementia (early onset Alzheimer's disease), senile dementia (dementia of the Alzheimer's type), Parkinsonism including Parkinson's disease, Huntington's chorea, tardive dyskinesia, hyperkinesia, mania, attention deficit disorder, anxiety, dyslexia, schizophrenia, psychosis, bipolar disorder, depression, and Tourette's syndrome, as well as epilepsy, sleep disorders, hearing and vision disorders, autism spectrum disorders and pain. Embodiments of the present disclosure can be used to discover therapeutic agents for the treatment of any of the aforementioned CNS disorders, as one of ordinary skill would readily appreciate based on the present disclosure.

In some embodiments, methods of the present invention include subjecting organisms to a battery of behavioral assays after exposing the organisms to an experimental perturbation. The experimental perturbation may exert an effect on the organisms which may be reflected in the behavioral assay and/or the behavioral profile. For example, behavioral assays can be conducted on organisms exposed to a chemical compound from a chemical library. In some cases, the chemical compounds used as experimental perturbations can have known or unknown biological functions. In some cases, the chemical compounds can have known biological functions, including but not limited to, psychoactive properties (e.g., haloperidol).

In addition to perturbations caused by chemical compounds, embodiments of the present disclosure can also be used to create behavioral profiles in the context of genetic or environmental perturbations. For example, materials and methods of the present disclosure can be used to create databases containing behavioral profiles of organisms with known or unknown genetic defects or mutations, which can be compared to the behavioral profiles of organisms with known genetic defects or mutations or wild type organisms, in an effort to link phenotypic similarities and identify a novel gene or genes contributing to a given phenotype. In some aspects, the underlying phenotype can mimic a human disease phenotype. In other aspects, the genetic mutation can be in a gene known to contribute to the disease phenotype in a human. Other experimental perturbations can include genetic mutations causing various quantifiable phenotypes, including transgenics, hypermorphs, hypomorphs, loss-of-function, and gain-of-function mutants. Mutations can occur at coding and non-coding loci. Behavioral assays can also be conducted to determine if chemical compounds are able to suppress a given phenotype induced by a genetic or environmental perturbation or another chemical compound (suppressor screen). Behavioral assays can also be created around various environmental perturbations (e.g., oxygen levels, relative pressures, degrees of heat/cold, radiation exposure, etc.).

Embodiments of the present invention also provide a high-throughput screening (HTS) platform for conducting the behavioral assays and generating quantifiable summaries of the behavioral profiles. HTS methods generally involve the use of robotics, data processing and control software, liquid handling devices, and sensitive detectors. The HTS methods of the present disclosure allow researchers to quickly conduct millions of biochemical, genetic or pharmacological assays and generate high-content data using whole organisms. The HTS platform of the present disclosure generally includes both hardware and software. The software is designed to control the hardware and analyze images captured of the behavior of the organisms during the behavioral assays. In some cases, the software synchronizes image acquisition with the presentation of one or more stimuli. The software can save, store, and organize the raw image data, extract behavioral data from the images and analyze the data for specific characteristics (e.g., common phenotypic characteristics). The software can also take these data and generate a quantitative summary or quantitative summaries of the behavioral profiles, which can be compared to another quantitative summary or other quantitative summaries, analyzed for similarities and differences, and stored in a database. A behavioral profile can be generated for each experimental perturbation (e.g., chemical or genetic). In some cases, the behavioral profiles (also known as behavioral barcodes) can be used to identify chemical compounds with a desired set of activities or biological effects, and also to predict biological mechanisms of action.

Integrated systems for use with the chemical screening platform of the present invention can comprise one or more automated devices for loading microtiter plates containing various organisms and/or detectable label(s), and/or test agent(s), and/or automated devices for reading the results of the assay. Integrated systems can also include additional robotics for sample processing, reagent synthesis, microtiter plate storage and/or incubation and/or handling, computer systems for controlling the devices and recording and/or analyzing assay data and the like. Other electronic hardware and/or software may be implemented to enhance image acquisition, data storage and/or synchronization, as one skilled in the art would appreciate from the present disclosure.

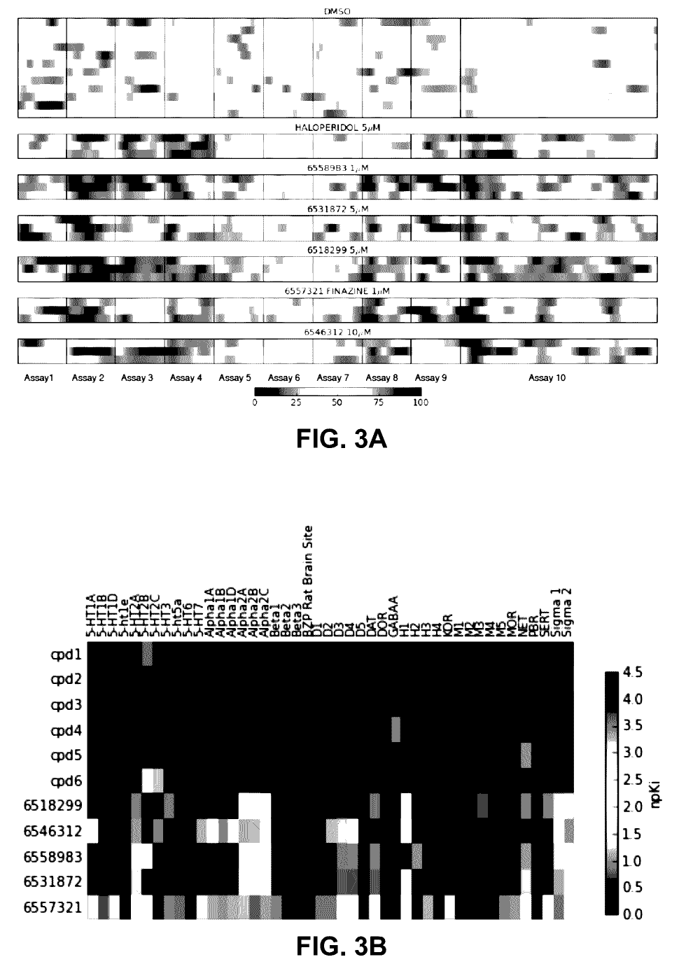

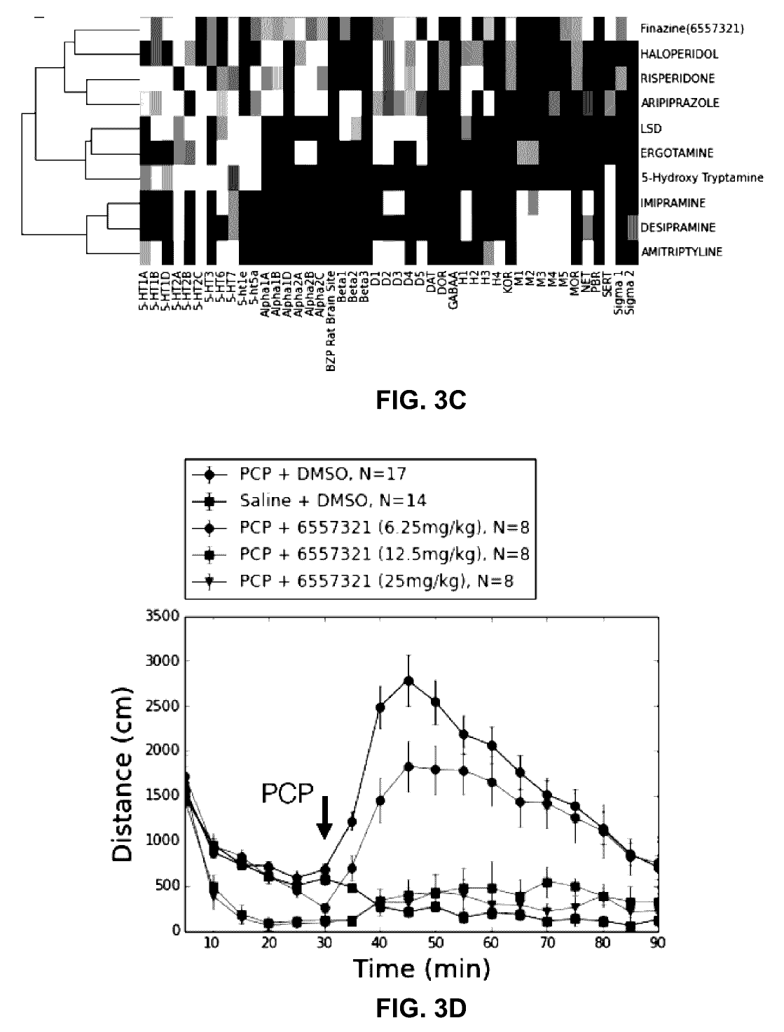

Once a phenotype-based chemical screen has been conducted, a quantitative summary or quantitative summaries corresponding to the behavioral profile(s) generated can be analyzed for common phenotypic characteristics. These data can be clustered to determine structural similarities among the chemical compounds identified as having, for example, similar behavioral profiles. In some cases, the data can be queried using a blast-type approach (e.g., phenoBlast) in that a reference compound with known biological functions can be compared to chemical compounds with unknown biological functions to identify those compounds with similar behavioral profiles. For example, the behavioral profile of haloperidol, a known therapeutic compound with antipsychotic effects in vertebrates, can be used to query a database comprised of the quantitative summaries of the behavioral profiles of other compounds. Compounds having similar behavioral profiles as haloperidol may also have similar antipsychotic effects in vertebrate animals (see, e.g., FIG. 3D). In some cases, the more behavioral assays making up a behavioral profile, the greater the sensitivity and power of the phenoBlast approach. In some cases, a database can include quantitative summaries of behavioral profiles corresponding to 20,000 or more chemical compounds.

Any organisms can be used with embodiments of the present disclosure provided the organisms are suited to high-throughput screening. Organisms well suited to high-throughput chemical screening include, but are not limited to, zebrafish larva (e.g., Danio rerio), fruit flies (e.g.,Drosophila melanogaster), and nematodes (e.g., C. elegans). The methods of the present disclosure are generally applicable for use in other teleosts as well. Suitable teleosts include, for example, Medaka, Giant rerio, and puffer fish. Organisms used can include organisms with various genetic perturbations, including, but not limited to, hypermorphs, hypomorphs, loss-of-function, and gain-of-function mutants. In some cases, transgenic organisms can be used in which various fluorescent molecules or fluorescently-tagged biomolecules can be quantified during behavioral assays.

Zebrafish embryos or zebrafish larva are well suited for conducting phenotype-based chemical screens. During the embryonic and larval stages of life, the zebrafish is only about 1-2 mm long, and can live for days in a single well of a standard 96-well or a standard 384-well plate, surviving on nutrients stored in its yolk sac. Chemical compounds can also easily diffuse and penetrate the embryonic and larval zebrafish body. The genome and body plan of the zebrafish are similar to those of other vertebrates, and its optical transparency and external development enable real time observation of its internal organs and physiological systems. The optical clarity of the zebrafish embryo becomes even more useful when combined with fluorescent markers that highlight the locations or activities of specific populations of cells. For example, dozens of transgenic zebrafish lines have been created which express fluorescent proteins in many different organs, tissues and cells in vivo. Transgenic zebrafish lines greatly facilitate detection of behavioral changes caused by small molecules. Numerous zebrafish disease models ranging from congenital heart defects to cancers have been developed, and the zebrafish is genetically and pharmacologically similar to humans. Further, because screening can be performed in the whole organism, perturbation of potential therapeutic targets by small molecules or mutations reveals the effects of such perturbations on the integrated physiology of the entire organism.

In some embodiments, zebrafish larva between about 2 days and 10 days post-fertilization (e.g., obtained by mating Ekkwill zebrafish) can be transferred to wells of clear, flat-bottom and square 96-well plates filled with embryo liquid medium (e.g., 300 μl of E3 medium) prior to entry of the plates into an automated high-throughput screening platform. In some cases, the stimuli applied during a behavioral assay as well as the digital recordings of the assay can be applied to the entire 96-well plate simultaneously. In some cases, each behavioral assay, including video recording and data processing, can occur in about 40s per plate (per 30s assay); a battery of 10 behavioral assays using 96-well plates can be screened in less than 10 minutes.

### EXAMPLES

Embodiments of the present invention are included to demonstrate certain embodiments presented herein. It should be appreciated by those of skill in the art that the techniques disclosed in the examples that follow represent techniques discovered to function well in the practices disclosed herein. However, those of skill in the art should, in light of the present disclosure, appreciate that many changes can be made in the certain embodiments which are disclosed and still obtain a like or similar result without departing from the spirit and scope herein.

### Example 1: Antipsychotic drugs cause complex behavioral phenotypes in zebrafish

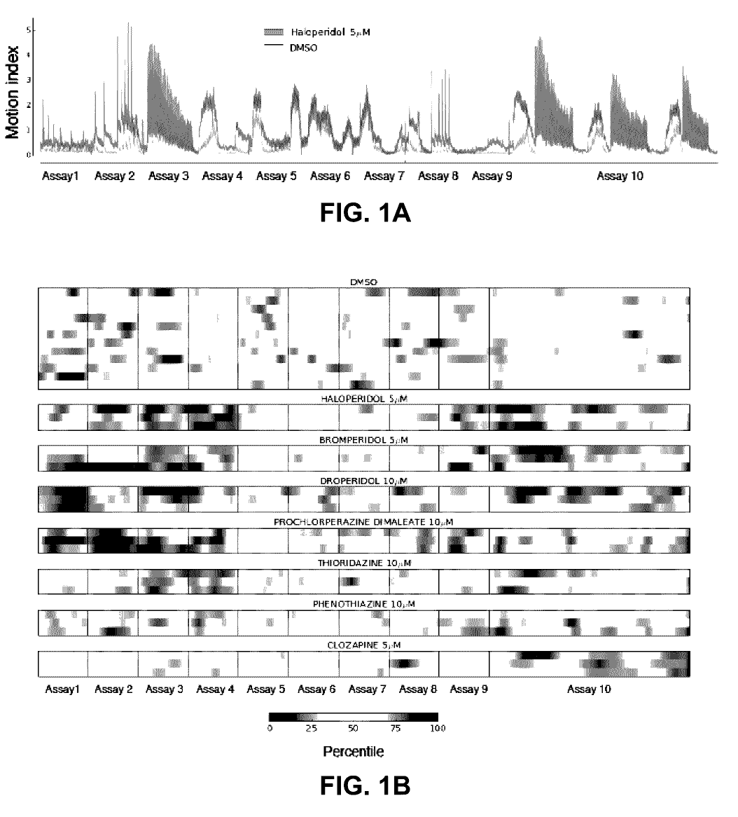

Haloperidol, a butyrophenone, is known to bind at least 20 molecular targets found in the human brain. To determine how haloperidol affects zebrafish behavior, a battery of ten behavioral assays was developed to compare behavioral profiles from control (DMSO) and haloperidol-treated animals. It was found that haloperidol caused complex and dose dependent effects on zebrafish behavior. At high concentrations (50 μM), haloperidol produced locomotor impairment in larval zebrafish, as previously reported. This is consistent with observations in mice. At lower concentrations (5 μM), haloperidol produced a complex pattern of increased activity, as measured by the motion index, which represents the collective motion in each well, as shown in FIG. 1A. The motion index was not greater in all assays (e.g., Assay 6 and Assay 7), and did not increase during baseline activity, suggesting haloperidol acts on specific pathways that control these complex patterns of behavior.

To test whether various antipsychotic drugs, including those structurally unrelated to butyrophenones, also cause haloperidol-like phenotypes, six additional compounds with antiphychotic activity were analyzed. It was found that five of the six also cause haloperidol-like behaviors including two additional butyrophenones (bromperidol and droperidol) and three phenothiazines (prochlorperazine, thioridazine, and phenothiazine), as shown in FIG. 1B. Clozapine, an atypical antipsychotic, did not cause a haloperidol-like stimulation phenotype at any concentration tested (FIG. 1B). Because multiple typical antipsychotic drugs from different structural classes (e.g., butyrophenones and phenothiazines) cause similar behavioral phenotypes, behavioral profiling in the zebrafish may enable a phenoBlast approach for identifying novel antipsychotic-like compounds.

These data establish a behavioral profile for haloperidol that is distinct from the profiles created by other neuroactive drugs, which can serve as a basis for performing phenotype-based database searches (e.g., phenoBlast) as well as phenotype-based chemical screens for novel haloperidol-like chemicals.

### Example 2: PhenoBlast methods identify novel haloperidol-like compounds

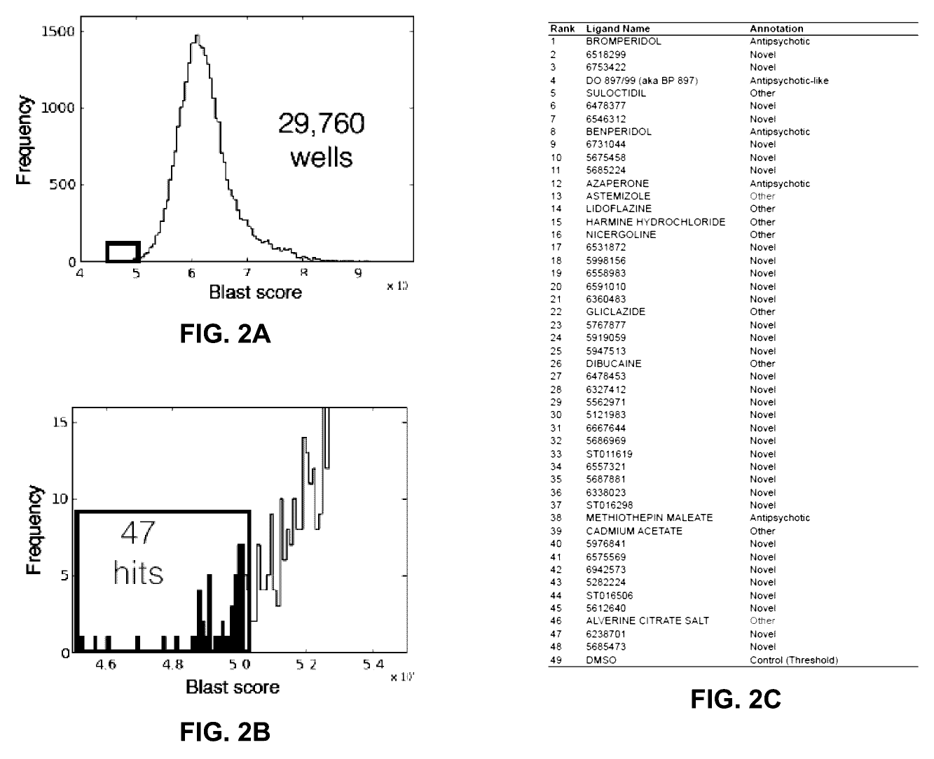

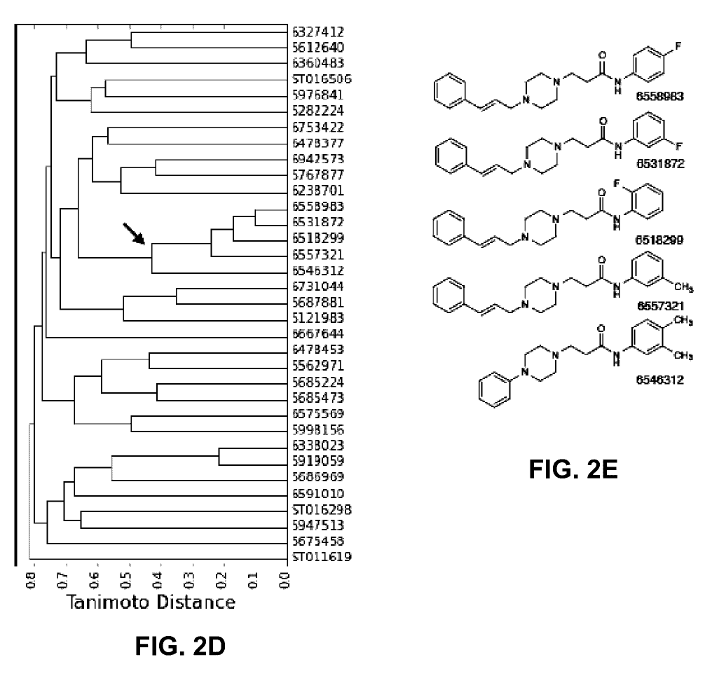

To identify compounds with haloperidol-like behavioral profiles, the behaviors of zebrafish larvae in 29,760 compound-treated wells (including 4,300 known bioactive compounds, 20,000 uncharacterized novel compounds and more than 5,000 DMSO controls) were systematically quantified. Behavioral profiles from each well were compiled into a database. Then, the average profile of three haloperidol (5 μM)-treated wells was used to query the compiled database for additional compounds that cause haloperidol-like phenotypes. Using the DMSO control well most similar to the haloperidol query as a cutoff, 47 hit compounds were identified that exhibited a higher degree of similarity to haloperidol than DMSO controls, as shown in FIGS. 2A-2B. The top ranked hit compound, bromperidol, is a close structural and functional analog of haloperidol, showing that the phenoBlast approach can identify antipsychotic compounds. Overall, fourteen hit compounds were annotated bioactive drugs, as shown in FIG. 2C. Four of the known hit compounds were antipsychotic drugs (including three butyrophenones and one tricyclic). A fifth compound, DO 897/99 has been under investigation for treatment of schizophrenia and a sixth compound, lidoflazine, is structurally related to the known antipsychotic amperozide and other antipsychotics in the diphenylbutylpiperidine class (e.g., clopimozide, fluspirilene and pimozide) (FIG. 2C).

Using the unbiased method of the phenoBLAST approach, both antipsychotic and antipsychotic-like compounds were identified among the top hits, suggesting that some of the thirty-three novel compounds also identified may have psychoactive properties as well, underscoring the utility of the methods of the present disclosure.

### Example 3: Finazines have bioactive properties

Novel hit compounds were clustered based on structural similarity (61%-89% Tanimoto similarity) to identify any potential structural relationships, as shown in FIG. 2D. A cluster of five closely related hit compounds were found that shared a piperazine-containing maximum common substructure. These compounds were termed “finazines” and are shown in FIG. 2E. Only thirty-eight compounds in the library of more than 20,000 contained the finazine substructure. To confirm that these compounds cause haloperidol-like phenotypes, all five of the finazine compounds were retested at seven different concentrations, as shown in FIG. 3A. The finazines reproducibly phenocopied the behavioral profiles of zebrafish exposed to haloperidol. At higher concentrations, like haloperidol, these compounds generally reduced motor activity (not shown). At lower concentrations (1-10 μM), these compounds caused haloperidol-like stimulation phenotypes (FIG. 3A).

These results demonstrate that the finazines represent a novel class of psychoactive chemicals that cause robust and reproducible haloperidol-like phenotypes in the zebrafish, and suggest that these psychoactive chemicals act via the same neurological pathways.

### Example 4: Hit compounds have polypharmacological, haloperidol-like binding profiles

Haloperidol exhibits activity at a variety of targets including dopaminergic, adrenergic and serotonergic receptors. Although dopamine blockade is an essential component of haloperidol's antipsychotic properties, the ability of haloperidol to affect multiple other signaling pathways is also considered important for its therapeutic activity in humans. To determine if, like haloperidol, the hit compounds identified by phenoBLAST also act on a variety of neurotransmitter signaling pathways, the binding affinity of the hit compounds for 45 candidate human and rodent receptors in vitro were tested. Like haloperidol, many of the hit compounds bound to a variety of receptors including serotonergic, alpha-adrenergic, and dopaminergic receptors, as shown in the representative heat map of FIG. 3. However, not all bioactive compounds exhibit polypharmacology at multiple receptors. For example, six unrelated novel bioactive compounds (cpd1-cpd6) from the same chemical libraries discovered using an unrelated zebrafish behavior failed to exhibit polypharmacology (FIG. 3B). The polycharmacology of representative finazine, 6557321, was also compared to nine other antipsychotic drugs, including antipsychotics, antidepressants and hallucinogens, using the same multi-receptor in vitro binding assay. Hierarchical clustering revealed that the nine other known antipsychotic drugs clustered with their annotated activities, and that finazine 6557321 clustered with haloperidol and the other antipsychotic compounds (FIG. 3C).

These results demonstrate that the phenoBLAST approach using, for example, haloperidol as a reference compound is an effective way to identify chemicals with polypharmacological binding profiles against receptors in the CNS. These data also suggest that, like haloperidol, finazine 6557321 may exhibit antipsychotic-like activity in mammals.

### Example 5: Finazines suppress PCP-induced hyperactivity in mice

Finazine 6557321 was tested to determine if it exhibits antipsychotic-like activity in a psychostimulant-induced schizophrenia mouse model. In humans, acute administration of psychostimulants such as phencyclidine (PCP), a NMDA receptor antagonist, induces psychosis-like symptoms in common with schizophrenia. In mice, PCP induces a hyperlocomotion phenotype that can be reversed by haloperidol and other typical and atypical antipsychotic drugs. To determine if finazine has antipsychotic-like activity in mammals three groups of mice were injected each with graded doses (25, 12.5 and 6.25 mg/kg). At the two higher doses the mice exhibited a decrease in baseline locomotor activity, similar to what was observed in the zebrafish and also to what is observed with higher doses of haloperidol in mice. Baseline locomotor activity was unaffected in mice treated with the lowest dose of finazine (6.25 mg/kg). Thirty minutes after compound treatment, mice were injected with PCP (5 mg/kg). Like haloperidol, finazine reduced the psychostimulant effect of PCP in the mouse model (FIG. 3D).

These data demonstrate that the antipsychotic-like activity of finazine 6557321 predicted using behavioral profile analysis in zebrafish was accurately recapitulated in both CNS receptor binding assays and in a psychostimulant-induced schizophrenia mouse model.

Although the above non-limiting examples relate to the use of materials and methods of the present disclosure to discover novel therapeutic agents to treat schizophrenia, embodiments of the present disclosure are applicable to the discovery of therapeutic agents for the treatment of any other CNS disorder, as one of ordinary skill would appreciate based on the present disclosure. Embodiments of the present disclosure can be used to discover novel therapeutic agents for the treatment of CNS disorders and diseases that include, but are not limited to, neuropsychiatric disorders, neurological diseases and mental illnesses, neurodegenerative diseases, behavioral disorders, cognitive disorders, and cognitive affective disorders, disorders whose clinical manifestations have been attributed to CNS dysfunction (e.g., disorders resulting from inappropriate levels of neurotransmitter release, inappropriate properties of neurotransmitter receptors, and or inappropriate interaction between neurotransmitters and neurotransmitter receptors), disorders attributed to a cholinergic deficiency, a dopaminergic deficiency, an adrenergic deficiency and/or a serotonergic deficiency, presenile dementia (early onset Alzheimer's disease), senile dementia (dementia of the Alzheimer's type), Parkinsonism including Parkinson's disease, Huntington's chorea, tardive dyskinesia, hyperkinesia, mania, attention deficit disorder, anxiety, dyslexia, schizophrenia, psychosis, bipolar disorder, depression and Tourette's syndrome, as well as epilepsy, sleep disorders, hearing and vision disorders, autism spectrum disorders and pain.

### Materials and Methods

### Aquaculture and Chemical Treatments

A large number of fertilized eggs (up to 20,000 embryos per day) were collected from group matings of Ekkwill zebrafish. Embryos were raised in hatching jars at 28 ° C. on a 14/10 hour light/dark cycle until 3 days post fertilization (d.p.f.), then transferred to an incubator under the same conditions until 7 d.p.f. Groups of approximately ten larvae (7 d.p.f.) were distributed into the wells of clear flat bottom square well 96-well plates filled with E3 medium (300 μl). Larvae were then incubated at 25 ° C. on the bench top for one hour prior to chemical treatment and subsequent experiments. All zebrafish protocols were approved by the Institutional Animal Care and Use Committee at Massachusetts General Hospital or at Teleos Therapeutics, LLC.

### Chemical Libraries and Treatments

The Actiprobe library (TimTec Corporation) contains 10,000 compounds dissolved in DMSO at a stock concentration of lmg/ml (˜3 mM). The Chembridge library (Chembridge Corporation) contains 10,000 compounds dissolved in DMSO at a stock concentration of 1 mM. The Spectrum Collection (MicroSource Discovery) contains 2,320 compounds dissolved in DMSO at a concentration of 10mM. The Prestwick library (Prestwick Chemical) contains 1,280 approved drugs dissolved in DMSO at a stock concentration of 10 mM. The Neurotransmitter library (Biomol International; cat. No. 2810) contains 700 compounds dissolved in DMSO at a stock concentration of 10 mM. All compounds were diluted in E3 buffer and screened at 10 μM final concentration and <1% DMSO. Negative controls were treated with an equal volume of DMSO. Stock solutions were added directly to zebrafish in the wells of a 96-well plate, mixed and allowed to incubate for 1 h at room temperature before behavioral evaluation in the Behavioral Battery of assays. Reordered hit compounds were dissolved in DMSO to a stock concentration of 30 mM and added to wells as described above. Hits were retested at a concentration range of 50, 10, 5, 1, 0.5, 0.1, and 0.05 μM in triplicate; assays for retesting were run at 10-minute and 1-hour incubation time points.

### Chemoinformatics

Instant JChem was used for structure database management and substructure searching, Instant JChem 14.7.14.0, 2014, ChemAxon. Chemical similarities were computed as Tanimoto values based on a Daylight-like fingerprinter using rdKit. CNS receptor profiling

For known psychoactive compounds, binding profiles were downloaded from the PDSP Ki database. Ki values that were greater than 10,000 nM or missing were set to 10,000 nM. Normalized Ki (npKi) values were computed as described. For novel hit compounds, in vitro binding assay and Ki data were generated by the National Institute of Mental Health's Psychoactive Drug Screening Program (PDSP), contract no. HHSN-271-2008-00025-C (NIMH PDSP).

### Mouse Phenotyping

Male C57BL/6J mice (9-10 weeks at testing) were obtained from Jackson Laboratories (Bar Harbor, Me.) Mice were group-housed 4 per cage in Techniplast ventilated cages and were maintained on a 12/12 hr light/dark cycle (lights on 0700 EST). The room temperature was maintained at 20-23° C. with relative humidity at approximately 50%. Food and water were available ad libitum for the duration of the study, except during testing and all testing was conducted during the light phase of the light dark cycle. The behavioral tests were conducted according to established protocols approved by the Harvard Medical Area (HMA) Standing Committee on Animals IACUC in AALAC-accredited facilities, and in accordance with the Guide to Care and Use of Laboratory Animals (National Institutes of Health 1996). Locomotor activity was measured in Plexiglas square chambers (27.3×27.3×20.3 cm; Med Associates Inc., St Albans, Vt.) surrounded by infrared photobeam sources and detectors. Mice were tested under ambient light and data were collected by Med Associates software. Mice were injected with 10% DMSO vehicle or finazine (6.25, 12.5, or 25 mg/kg in 10% DMSO) and locomotor activity was monitored for 30 m (baseline total distance). Mice were then administered saline vehicle or PCP (5 mg/kg) and activity was measured for an additional 60 m. Antagonism of PCP-induced hyperactivity was used as the measure of antipsychotic efficacy. All compounds were administered by intraperitoneal (IP) injection in a volume of 10 ml/kg. Locomotor activity was measured as total distance traveled (cm), assessed via infrared beam breaks. Locomotion prior to PCP administration (baseline, 0-30 m) and locomotion post PCP administration (PCP, 30-60 m) were analyzed by one-way analysis of variance (ANOVA) with finazine (0, 6.25, 12.5, 25) as the independent variable. All significant effects were followed up with the Fisher's PLSD post hoc test. An effect was considered significant if p<0.05 (Statview for Windows, Version 5.0).

### Automated Behavioral Phenotyping in Zebrafish

Digital video were captured at 25 frames per second using an AVT Pike digital camera. High intensity LEDs were used to deliver violet (405 nm), blue (450 nm), and red (660 nm) light stimuli. Acoustic stimuli were delivered using push-style solenoids to tap the stage. Stimuli and digital recordings were applied to the entire 96-well plate simultaneously. Instrument control and data acquisition were performed using custom MATLAB scripts. Each assay, including video recording and data processing, took ˜40s per plate per 30s assay, enabling us to routinely screen 96 wells against a battery of ten assays in less than ten minutes. To analyze digital video recordings, a motion index was calculated by frame differencing. This motion index correlates with the overall amount of motion in the well. The behavioral battery was comprised of ten assays (Assay1-10) performed in numerical order. phenoBlast ranking and statistics

For each well, the raw motion index from each assay was concatenated into a 10,500-point motion index time series. This raw motion index was normalized by subtracting the 5^{th} percentile of that time series. The haloperidol query was calculated by averaging the time series from three replicate haloperidol-treated wells. To calculate the phenoBLAST distance metric, L1 distances were calculated by: phenoBLAST Distance=sum (abs (query-well)), where ‘query’ and ‘well’ indicate their associated time series.

It should also be appreciated that the exemplary embodiment or exemplary embodiments are only examples, and are not intended to limit the scope, applicability, or configuration of the subject matter hereof in any way. Rather, the foregoing detailed description will provide those skilled in the art with an enabling disclosure for implementing the exemplary embodiment or exemplary embodiments. It should be understood that various changes can be made in the function and arrangement of elements without departing from the scope of the subject matter hereof as set forth in the appended claims and the legal equivalents thereof.

The embodiments above are intended to be illustrative and not limiting. Additional embodiments are within the claims. Although the present subject matter has been described with reference to particular embodiments, workers skilled in the art will recognize that changes may be made in form and detail without departing from the spirit and scope of the subject matter hereof.

Various modifications to the subject matter hereof may be apparent to one of skill in the art upon reading this disclosure. For example, persons of ordinary skill in the relevant art will recognize that the various features described for the different embodiments of the subject matter can be suitably combined, un-combined, and re-combined with other features, alone, or in different combinations, within the spirit of the subject matter hereof. Likewise, the various features described above should all be regarded as example embodiments, rather than limitations to the scope or spirit of the subject matter hereof. Therefore, the above is not contemplated to limit the scope of the present subject matter hereof.

The term “means” as used herein shall be given its broadest possible interpretation in accordance with 35 U.S.C. §112(f). Accordingly, a claim incorporating the term “means” shall cover all structures, materials, or acts set forth herein, and all of the equivalents thereof. Further, the structures, materials or acts and the equivalents shall include all those described in the summary, brief description of the drawings, detailed description, abstract, and claims themselves.

Appendix A is incorporated herein by reference in its entirety and for all purposes.

All of the MATERIALS and METHODS disclosed and claimed herein can be made and executed without undue experimentation in light of the present disclosure. While the compositions and methods have been described in terms of preferred embodiments, it is apparent to those of skill in the art that variations maybe applied to the MATERIALS and METHODS and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

What is claimed:

1. A method for identifying novel therapeutic chemical compounds or assessing properties of a known chemical compounds, the method comprising:
comparing a quantitative summary of a behavioral profile of a known bioactive chemical compound to a database of quantitative summaries of behavioral profiles; and
identifying one or more chemical compounds from the database of quantitative summaries of behavioral profiles based on phenotypic similarities to the behavior profile of the known bioactive chemical compound.

2. The method of claim 1, wherein the quantitative summaries of behavioral profiles are generated using one or more organisms.

3. The method of claim 1, wherein the behavioral profile is comprised of one or more behavioral assays, and wherein the one or more behavioral assays comprise the presentation of one or more stimuli.

4. The method of claim 3, wherein the one or more stimuli comprises one or more of light, sound, vibration, visual patterns, and electrical shocks.

5. The method of claim 1, wherein the known bioactive chemical compound has psychoactive properties.

6. The method of claim 1, wherein the method is performed as part of an automated high-throughput screening platform.

7. A method for performing a high-throughput screen for bioactive chemical compounds, the method comprising:
exposing at least one organism to a chemical compound;
subjecting the at least one organism to one or more behavioral assays and generating a behavioral profile; and
obtaining a quantitative summary of the behavioral profile and storing the quantitative summary in a database.

8. The method of claim 7, further comprising:
repeating the exposing, the subjecting, the obtaining, and the storing steps using a plurality of chemical compounds to generate a database of quantitative summaries of behavioral profiles; and
analyzing the database of quantitative summaries of behavioral profiles for common phenotypic characteristics.

9. The method of claim 7, wherein the at least one organism is a zebrafish, a fruit fly, or a nematode.

10. The method of claim 7, wherein the one or more behavioral assays comprises the presentation of one or more stimuli.

11. The method of claim 10, wherein the one or more stimuli comprises one or more of light, sound, vibration, visual patterns, and electrical shocks.

12. The method of claim 7, wherein the plurality of chemical compounds comprise chemical compounds with psychoactive properties.

13. The method of claim 7, wherein the method is performed using an automated high-throughput screening platform.

14. The method of claim 7, wherein the method is perfumed using an automated high-throughput screening platform comprising software that synchronizes image acquisition when subjecting the at least one organism to the one or more behavioral assay, and extracts behavioral data when obtaining the quantitative summary of the behavioral profile.

15. A system for performing a high-throughput screen for bioactive chemical compounds, the system comprising:
a digital video camera capable of capturing and storing images;
a mechanism for presenting one or more stimuli to at least one organism when subjecting the at least one organism to one or more behavioral assays; and
software that synchronizes the capturing and storing of images when subjecting the at least one organism to one or more behavioral assays to generate one or more behavioral profiles, and extracts behavioral data from the one or more behavioral profiles to generate a one or more quantitative summaries.

16. The system of claim 15, further comprising a mechanism for exposing the at least one organism to one or more chemical compounds, wherein the at least one organism comprises a larval zebrafish, a fruit fly, or nematode.

17. The system of claim 15, wherein the one or more stimuli comprises one or more of light, sound, vibration, visual patterns, and electrical shocks.

18. The system of claim 15, wherein the one or more stimuli comprises light at wavelengths of at least 405 nanometers, 450 nanometers, and 660 nanometers.

19. A method for identifying a novel genetic or environmental perturbation, the method comprising:
comparing a quantitative summary of a behavioral profile corresponding to a genetic or environmental perturbation to a database of quantitative summaries of behavioral profiles; and
identifying phenotypic similarities between the quantitative summary of a behavior profile corresponding to a genetic or environmental perturbation and one or more quantitative summaries of behavioral profiles from the database of quantitative summaries of behavioral profiles.

20. A method for performing a high-throughput screen, the method comprising:
providing at least one organism having a phenotype based on a genetic or environmental perturbation;
subjecting the at least one organism to one or more behavioral assays and generating a behavioral profile; and
obtaining a quantitative summary of the behavioral profile and storing the quantitative summary in a database.

21. The method of claim 20, further comprising:
repeating the providing, the subjecting, the obtaining, and the storing steps using a plurality of organisms having phenotypes based on genetic or environmental perturbations to generate a database of quantitative summaries of behavioral profiles; and
analyzing the database of quantitative summaries of behavioral profiles for common phenotypic characteristics.
